(19) United States
(12) Patent Application Publication
(10) Pub. No.: US 2025/0288778 A1
(43) Pub. Date: Sep. 18, 2025

(54) MECHANISM FOR COUPLING A DILATOR AND SHEATH

(71) Applicant: Boston Scientific Scimed, Inc., Maple Grove, MN (US)

(72) Inventor: Jeffery D. Arnett, Louisa, VA (US)

(21) Appl. No.: 19/081,894

(22) Filed: Mar. 17, 2025

Related U.S. Application Data
(60) Provisional application No. 63/566,460, filed on Mar. 18, 2024.

(51) Int. Cl. A61M 25/06; A61M 25/00; A61M 29/00

(52) U.S. Cl. CPC A61M 25/0662; A61M 25/0097; A61M 29/00; A61M 2205/6045; A61M 2210/125

(57) ABSTRACT

A system for facilitating access to a patient's heart includes a sheath having a sheath body defining a lumen adapted to receive a dilator. The sheath body includes a proximal portion and a distal portion. A sheath hub is coupled to the proximal portion of the sheath. The sheath hub includes an opening. The system includes a dilator having a dilator shaft defining a lumen adapted to receive and support a puncturing device. The dilator shaft includes a proximal portion for manipulation by a user and a tapered distal portion for placement in or near the heart. A dilator hub is coupled to the proximal portion of the dilator shaft. The dilator hub includes a flange configured to be received in the opening. The opening and the flange include a symmetric shape along a vertical axis and an asymmetric shape along a horizontal axis.

### CROSS REFERENCE TO RELATED APPLICATIONS

This application claims priority to U.S. Provisional Patent Application No. 63/566,460 entitled “MECHANISM COUPLING A DILATOR AND SHEATH,” filed Mar. 18, 2024, which is incorporated herewith by reference in its entirety.

### TECHNICAL FIELD

The present invention relates generally to methods and devices usable to deliver a therapy to a patient. More specifically, the present invention is concerned with a system and method for delivering a therapy device to a heart.

### BACKGROUND

Devices currently exist for creating a puncture, channel, or perforation within a tissue located in a body of a patient. One such device is the Brockenbrough™ Needle, which is commonly used to puncture the atrial septum of the heart. This device is a stiff elongated needle, which is structured such that it may be introduced into a body of the patient via the femoral vein, and directed towards the heart. This device relies on the use of mechanical force to drive the sharp tip through the septum. Alternatively, radiofrequency perforation apparatuses have been developed, whereby the septal perforation is accomplished by the application of focused radiofrequency energy to the septal tissue via an electrode at the distal end of a relatively thin conductive probe.

Such perforation devices are often used in conjunction with a dilator to help support and guide the perforation device. Such dilators are often used in conjunction with a therapy sheath adapted to deliver a therapy to the patient.

### SUMMARY

Example 1 is a dilator for facilitating access to a patient's heart and for coupling with a sheath including a sheath hub. The dilator includes a dilator shaft defining a lumen adapted to receive and support a puncturing device. The dilator shaft includes a proximal portion for manipulation by a user and a distal portion for placement in or near the heart. A dilator hub is coupled to the proximal portion of the dilator shaft. The dilator hub including a flange having a symmetric shape along a vertical axis and an asymmetric shape along a horizontal axis. The flange is configured for coupling to the sheath hub to inhibit relative rotation between the dilator and the sheath.

Example 2 is the dilator of Example 1, wherein the dilator hub is directly coupled to the dilator shaft.

Example 3 is the dilator of Examples 1 or 2, wherein the sheath is a therapy sheath and the puncturing device is an RF puncturing device.

Example 4 is the dilator of any of Examples 1-3, further comprising an axial lock feature configured to create a resistance to an axial disengagement force, such that the dilator hub is secured axially within the sheath hub.

Example 5 is the dilator of any of Examples 1-4, wherein the axial lock feature includes a protrusion adapted to mate with a shoulder.

Example 6 is the dilator of Example 5, wherein the protrusion is annular.

Example 7 is the dilator of any of Examples 5 or 6, wherein the protrusion includes a distal angled surface, a proximal angled surface, and an outer flat surface located between the distal angled surface and the proximal angled surface.

Example 8 is the dilator of any of Examples 1-7, wherein the hub includes a cylindrical portion, the axial lock feature being located at a distal end of the cylindrical portion.

Example 9 is the dilator of any of Examples 1-8, wherein the flange includes a thickness that is angled towards the distal portion.

Example 10 is the dilator of any of Examples 1-9, wherein the flange includes a surface that includes a plurality of supports.

Example 11 is the dilator of Example 10, wherein the plurality of supports include a bottom support, a middle support, and an upper support.

Example 12 is the dilator of Example 11, wherein the upper support includes a first horizontal support and a second vertical support, the second vertical support being aligned with the vertical axis.

Example 13 is the dilator of any of Examples 1-10, wherein the flange includes a horizontal bottom wall, a left wall substantially orthogonal to the horizontal bottom wall, a right wall substantially orthogonal to the horizontal bottom wall, and a curved upper wall.

Example 14 is the dilator of any of Examples 1-10, wherein the flange includes a first wall perpendicular to a pair of spaced apart second walls.

Example 15 is the dilator of Example 12, wherein a third convex wall is opposite the first wall and joins the pair of spaced apart second walls.

Example 16 is a dilator for facilitating access to a patient's heart and for coupling with a sheath including a sheath hub. The dilator includes a dilator shaft defining a lumen adapted to receive and support a puncturing device. The dilator shaft includes a proximal portion for manipulation by a user and a tapered distal portion for placement in or near the heart. A dilator hub is coupled to the proximal portion of the dilator shaft. The dilator hub includes a flange having a symmetric shape along a vertical axis and an asymmetric shape along a horizontal axis. The flange is configured for coupling to the sheath hub to inhibit relative rotation between the dilator and the sheath.

Example 17 is the dilator of Example 16, further comprising an axial lock feature configured to create a resistance to an axial disengagement force, such that the dilator hub is secured axially within the sheath hub.

Example 18 is the dilator of Example 17, wherein the axial lock feature includes an annular protrusion adapted to mate with a shoulder.

Example 19 is the dilator of Example 18, wherein the protrusion includes a distal angled surface, a proximal angled surface, and an outer flat surface located between the distal angled surface and the proximal angled surface.

Example 20 is the dilator of Example 16, wherein the hub includes a cylindrical portion, the axial lock feature being located at a distal end of the cylindrical portion.

Example 21 is the dilator of Example 16, wherein the flange includes a thickness that is angled towards the distal portion.

Example 22 is the dilator of Example 16, wherein the flange includes a surface that includes a plurality of supports.

Example 23 is the dilator of Example 16, wherein the flange includes a horizontal bottom wall, a left wall substantially orthogonal to the horizontal bottom wall, a right wall substantially orthogonal to the horizontal bottom wall, and a curved upper wall.

Example 24 is the dilator of Example 16, wherein the flange includes a first wall perpendicular to a pair of spaced apart second walls, and a third convex wall opposite the first wall.

Example 25 is a sheath for facilitating access to a patient's heart and for coupling with a dilator including a dilator hub. The sheath includes a sheath shaft defining a lumen adapted to receive and support a dilator. The sheath shaft includes a proximal portion for manipulation by a user and a distal opening through with the dilator can extend. A sheath hub is coupled to the proximal portion of the sheath shaft. The sheath hub includes an opening having a symmetric shape along a vertical axis and an asymmetric shape along a horizontal axis. The opening is configured for coupling to a flange of the dilator to inhibit relative rotation between the sheath and the dilator.

Example 26 is the sheath of Example 25, wherein the opening includes a horizontal bottom wall, a left wall substantially orthogonal to the horizontal bottom wall, a right wall substantially orthogonal to the horizontal bottom wall, and a curved upper wall.

Example 27 is the sheath of Example 25, wherein the opening includes a first wall perpendicular to a pair of spaced apart second walls, and a third convex wall opposite the first wall.

Example 28 is the sheath of Example 25, wherein the sheath hub includes a passageway to receive at least a portion of a dilator hub.

Example 29 is the sheath of Example 25, wherein the sheath hub includes a recess to receive a valve.

Example 30 is the sheath of Example 29, wherein the sheath hub includes at least one connector, and a valve plate configured to receive a snaps racetrack, wherein the valve plate includes a plurality of rails to guide a hub cap and a protrusion.

Example 31 is the sheath of Example 30, wherein the protrusion prevents incorrect orientation of the hub cap.

Example 32 is the sheath of Example 31, wherein the hub cap includes a plurality of slots to receive the plurality of rails, and at least one opening to receive the connector.

Example 33 is a system for facilitating access to a patient's heart. The system includes a sheath having a sheath body defining a lumen adapted to receive a dilator. The sheath body includes a proximal portion and a distal portion. A sheath hub is coupled to the proximal portion of the sheath. The sheath hub includes an opening. The system includes a dilator having a dilator shaft defining a lumen adapted to receive and support a puncturing device. The dilator shaft includes a proximal portion for manipulation by a user and a tapered distal portion for placement in or near the heart. A dilator hub is coupled to the proximal portion of the dilator shaft. The dilator hub includes a flange configured to be received in the opening. The opening and the flange include a symmetric shape along a vertical axis and an asymmetric shape along a horizontal axis.

Example 34 is the system of Example 33, wherein the opening and the flange include a horizontal bottom wall, a left wall substantially orthogonal to the horizontal bottom wall, a right wall substantially orthogonal to the horizontal bottom wall, and a curved upper wall.

Example 35 is the system of Example 33, wherein the opening and the flange include a first wall perpendicular to a pair of spaced apart second walls, and a third convex wall opposite the first wall.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIGS. 1A-1C are schematic illustrations of a medical procedure within a patient's heart utilizing a transseptal access system according to embodiments of the invention.

FIG. 2 is a perspective view of a dilator being inserted into a sheath hub, according to embodiments of the invention.

FIG. 3 is a perspective view of the dilator hub of FIG. 2, according to embodiments of the invention.

FIG. 4 is a perspective view of the sheath hub of FIG. 2, according to embodiments of the invention.

FIG. 5 is an exploded view of the sheath hub of FIG. 2, according to embodiments of the invention.

FIG. 6A is a side view of the sheath hub of FIG. 2 illustrating a cap in a correct orientation, and FIG. 6B is a side view of the sheath hub of FIG. 2 illustrating a cap in an incorrect orientation, according to embodiments of the invention.

FIG. 7 is a cross-sectional view of the sheath hub of FIG. 2, according to embodiments of the invention.

FIG. 8 is a cross-sectional view of the sheath hub of FIG. 2 mated with a dilator hub, according to embodiments of the invention.

While the invention is amenable to various modifications and alternative forms, specific embodiments have been shown by way of example in the drawings and are described in detail below. The intention, however, is not to limit the invention to the embodiments described. On the contrary, the invention is intended to cover all modifications, equivalents, and alternatives falling within the scope of the invention as defined by the appended claims.

### DETAILED DESCRIPTION

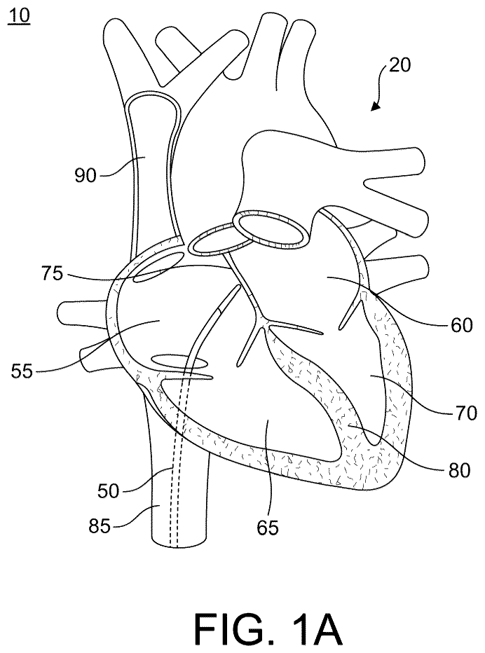

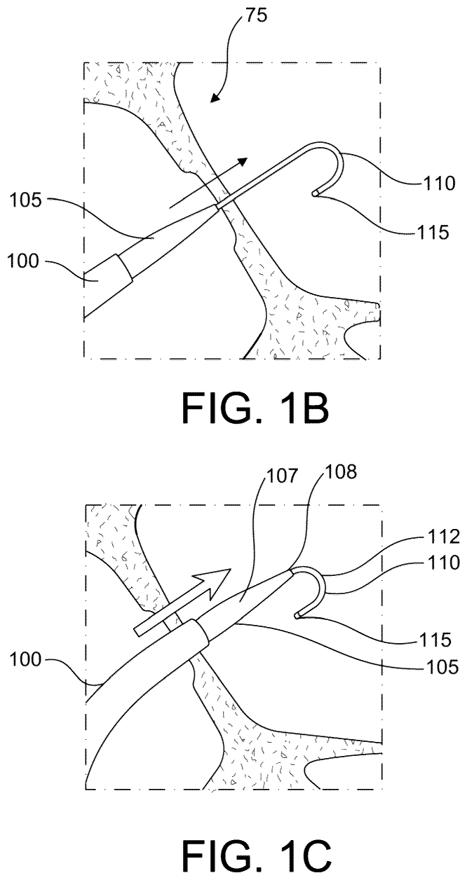

FIGS. 1A-1C are schematic illustrations of a medical procedure 10 within a patient's heart 20 utilizing a transseptal access system 50 according to embodiments of the disclosure. As is known, the human heart 20 has four chambers, a right atrium 55, a left atrium 60, a right ventricle 65 and a left ventricle 70. Separating the right atrium 55 and the left atrium 60 is an atrial septum 75 and separating the right ventricle 65 and the left ventricle 70 is a ventricular septum 80. As is further known, deoxygenated blood from the patient's body is returned to the right atrium 55 via an inferior vena cava (IVC) 85 or a superior vena cava (SVC) 90.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

The medical procedure 10 illustrated in FIGS. 1A-1C is an exemplary embodiment for providing access to the left atrium 60 using the transseptal access system 50 for subsequent deployment of the aforementioned diagnostic and/or therapeutic devices within the left atrium 60. As shown in FIGS. 1A-1C, target tissue site can be defined by tissue on the atrial septum 75. In the illustrated embodiment, the target site is accessed via the IVC 85, for example through the femoral vein, according to conventional catheterization techniques. In other embodiments, access to the target site on the atrial septum 75 may be accomplished using a superior approach wherein the transseptal access system 50 is advanced into the right atrium 55 via the SVC 90.

In the illustrated embodiment, the transseptal access system 50 includes an introducer sheath 100, a dilator 105 having a dilator body 107 and a tapered distal tip portion 108, and a perforation device (e.g., a radiofrequency (RF) perforation device) 110 having distal end portion 112 terminating in a tip electrode 115. As shown, in the assembled use state illustrated in FIGS. 1A-1C, the RF perforation device 110 can be disposed within the dilator 105, which itself can be disposed within the sheath 100. In one embodiment in which the transseptal access system 50 is deployed into the right atrium 55 via the IVC 105, a user introduces a guidewire (not shown) into a femoral vein, typically the right femoral vein, and advances it towards the heart 20. The sheath 100 may then be introduced into the femoral vein over the guidewire, and advanced towards the heart 20. In one embodiment, the distal ends of the guidewire and sheath 100 are then positioned in the SVC 90. These steps may be performed with the aid of an imaging system, e.g., fluoroscopy or ultrasonic imaging. The dilator 105 may then be introduced into the sheath 100 and over the guidewire, and advanced through the sheath 100 into the SVC 90. Alternatively, the dilator 105 may be fully inserted into the sheath 100 prior to entering the body, and both may be advanced simultaneously towards the heart 20. When the guidewire, sheath 100, and dilator 105 have been positioned in the SVC 90, the guidewire is removed from the body, and the sheath 100 and the dilator 105 are retracted so that their distal ends are positioned in the right atrium 55. The RF perforation device 110 described can then be introduced into the dilator 105, and advanced toward the heart 20. In various embodiments, the guidewire and the RF perforation device are the same component, so an exchange is not necessary.

Subsequently, the user may position the distal end of the dilator 105 against the atrial septum 75, which can be done under imaging guidance. The RF perforation device 110 is then positioned such that electrode 115 is aligned with or protruding slightly from the distal end of the dilator 105. The dilator 105 and the RF perforation device 110 may be dragged along the atrial septum 75 and positioned, for example against the fossa ovalis of the atrial septum 75 under imaging guidance. A variety of additional steps may be performed, such as measuring one or more properties of the target site, for example an electrogram or ECG (electrocardiogram) tracing and/or a pressure measurement, or delivering material to the target site, for example delivering a contrast agent. Such steps may facilitate the localization of the tip electrode 115 at the desired target site. In addition, tactile feedback provided by medical RF perforation device 110 is usable to facilitate positioning of the tip electrode 115 at the desired target site.

With the tip electrode 115 and dilator 105 positioned at the target site, energy is delivered from an energy source, e.g., an RF generator, through the RF perforation device 110 to the tip electrode 115 and the target site. In some embodiments, the energy is delivered at a power of at least about 5 W at a voltage of at least about 75 V (peak-to-peak), and functions to vaporize cells in the vicinity of the tip electrode 115, thereby creating a void or perforation through the tissue at the target site. The user then applies force to the RF perforation device 110 so as to advance the tip electrode 115 at least partially through the perforation. In these embodiments, when the tip electrode 115 has passed through the target tissue, that is, when it has reached the left atrium 60, energy delivery is stopped. In some embodiments, the step of delivering energy occurs over a period of between about 1 second and about 5 seconds.

With the tip electrode 115 of the RF perforation device 110 having crossed the atrial septum 75, the dilator 105 can be advanced forward, with the tapered distal tip portion 108 operating to gradually enlarge the perforation to permit advancement of the distal end of the sheath 100 into the left atrium 60.

In some embodiments, the distal end portion 112 of the RF perforation device 110 may be pre-formed to assume an atraumatic shape such as a J-shape (as shown in FIGS. 1B-1C), a pigtail shape or other shape selected to direct the tip electrode 115 away from the endocardial surfaces of the left atrium 60. Examples of such RF perforation devices can be found, for example, in U.S. patent application Ser. Nos. 16/445,790 and 16/346,404 assigned to Baylis Medical Company, Inc. The aforementioned pre-formed shapes can advantageously function to minimize the risk of unintended contact between the tip electrode 115 and tissue within the left atrium 60, and can also operate to anchor the distal end portion 112 within the left atrium 60 during subsequent procedural steps. For example, in embodiments, the RF perforation device 110 can be structurally configured to function as a delivery rail for deployment of a relatively larger bore therapy delivery sheath and associated dilator(s). In such embodiments, the dilator 105 and the sheath 100 are withdrawn following deployment of the distal end portion 112 of the RF perforation device 110 into the left atrium 60. The anchoring function of the pre-formed distal end portion 112 inhibits unintended retraction of the distal end portion 112, and corresponding loss of access to the perforated site on the atrial septum 75, during such withdrawal.

Various medical procedures have been developed for diagnosing or treating physiological ailments originating within the left atrium 60 and associated structures. Exemplary such procedures include, without limitation, deployment of diagnostic or mapping catheters within the left atrium 60 for use in generating electroanatomical maps or diagnostic images thereof. Other exemplary procedures include endocardial catheter-based ablation (e.g., radiofrequency ablation, pulsed field ablation, cryoablation, laser ablation, high frequency ultrasound ablation, and the like) of target sites within the chamber or adjacent vessels (e.g., the pulmonary veins and their ostia) to terminate cardiac arrythmias such as atrial fibrillation and atrial flutter. Still other exemplary procedures may include deployment of left atrial appendage (LAA) closure devices. Of course, the foregoing examples of procedures within the left atrium 60 are merely illustrative and in no way limiting with respect to the present disclosure.

In certain embodiments, catheters, therapy devices and sheaths can be deployed through the sheath 100, after it is successfully deployed into the desired heart chamber (e.g., the left atrium). In other embodiments, the therapy device (e.g., mapping catheter, therapy sheath, medical device, etc.) is part of the sheath 100, creating a therapy sheath.

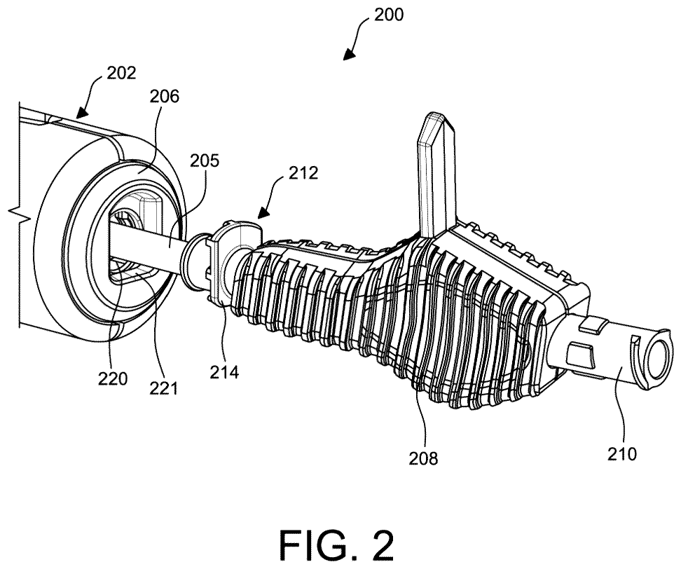

FIG. 2 is a perspective view of a system 200 for facilitating access to a patient's heart. The system includes a dilator 204 with a dilator hub 212 configured for insertion into a sheath 202 having a sheath hub 206, according to embodiments of the invention. As shown, the dilator 204 is partially inserted into a lumen of the sheath 202. The dilator 205 includes a handle 208, a dilator hub 212, a proximal connector 210, and a dilator shaft 205.

The sheath 202 includes a sheath hub 206 and a sheath shaft (not shown) defining a lumen adapted to receive and support the dilator shaft 205. The sheath shaft includes a proximal portion for manipulation by a user and a distal opening through with the dilator can extend as discussed above in FIGS. 1A-10. The sheath hub 206 is coupled to the proximal portion of the sheath shaft. The sheath hub 206 can be directly coupled or indirectly coupled to the sheath shaft. The sheath hub 206 includes a passageway 220 for receiving a portion of the dilator hub. The sheath hub 206 includes an opening 221 surrounding and leading to the passageway 220. The opening 221 has a symmetric shape along a vertical axis and an asymmetric shape along a horizontal axis. The opening 221 is configured for coupling to a flange 214 of the dilator 204 to inhibit relative rotation between the sheath 202 and the dilator 204.

The dilator 204 includes a dilator shaft 205 defining a lumen adapted to receive and support a puncturing device. A puncturing device can be inserted into the dilator 204 using the proximal connector 210. The proximal connector 210 is also configured to connect to a fluid introducing apparatus such as a syringe or other container for delivering fluid, such as a contrast medium, through the dilator shaft 205. The puncturing device can include a mechanical perforation device, for example a guidewire having a needle tip, or an RF puncturing device. The dilator shaft 205 includes a proximal portion for manipulation by a user, for example by using handle 208, and a tapered distal portion for placement in or near the heart. A dilator hub 212 is coupled to the proximal portion of the dilator shaft 205. The dilator hub 212 can be directly coupled or indirectly coupled to the dilator shaft 205. The dilator hub 212 includes a flange 214 having a shape that is substantially identical to the opening 221. The flange 214 includes a symmetric shape along a vertical axis and an asymmetric shape along a horizontal axis. When the flange 214 is received in the opening 221, relative rotation between the dilator 204 and the sheath 202 is inhibited or prevented. This creates a rotational lock between the two components. This allows a user to simultaneously rotate the dilator 204 and sheath 202 together.

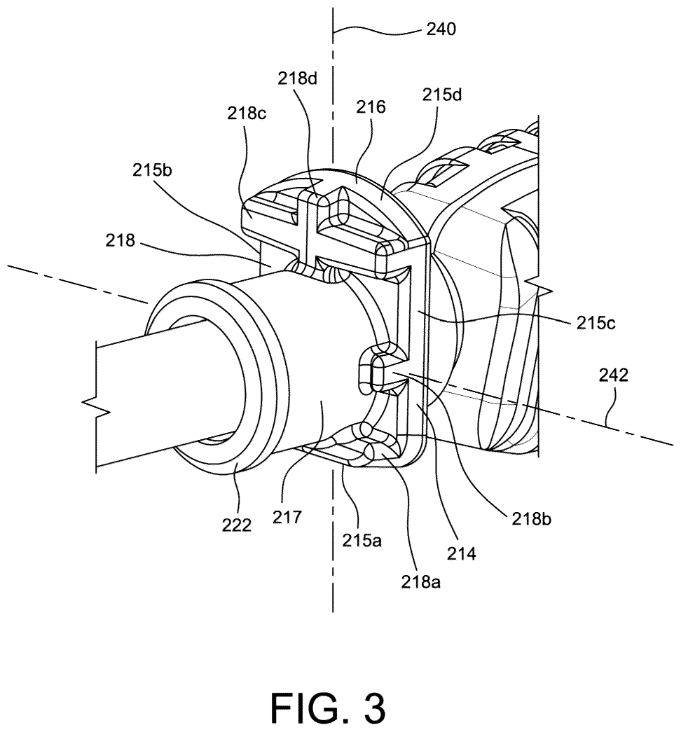

FIG. 3 is a perspective view of the dilator hub 212 of FIG. 2, according to embodiments of the invention. The flange 214 of the dilator hub 212 includes a symmetric shape along a vertical axis 240 and an asymmetric shape along a horizontal axis 242. The vertical axis 240 and the horizontal axis 242 pass through a longitudinal axis of the dilator 204. The flange 214 includes an edge 216 having a thickness that is angled towards the distal portion. In other words, the edge 216 tapers towards the distal portion of the dilator 204. This allows for the flange 214 to easily self-align into the opening. In some embodiments, the edge 216 does not taper, but is substantially parallel to a longitudinal axis of the dilator hub 212.

The flange 214 includes a surface 218 that includes a plurality of supports 218a, 218b, 28c, 218d. The plurality of supports include a bottom support 218a, a middle support 218b, and an upper support 218c, 218c. The upper support includes a first horizontal support 218c and a second vertical support 218d, the second vertical support 218d is aligned with the vertical axis 240. In some embodiments, the surface 218 can be a distal surface of the flange 214. In other embodiments, the supports can be placed on a proximal surface of the flange 214.

The flange 214 includes a horizontal bottom wall 215a, a left wall 215b substantially orthogonal to the horizontal bottom wall 215a, a right wall 215b substantially orthogonal to the horizontal bottom wall 215a, and a curved upper wall 215d. In other words, the flange 214 includes a first wall 215a perpendicular to a pair of spaced apart second walls 215b, 215c, and a third convex or domed wall 215d opposite the first wall 215a that joins the pair of spaced apart second walls 215b, 215c. It is not necessary for the shape of the flange 214 to match the shape of the opening 221 identically. For example, in some embodiments, the curved upper wall 215d is replaced by a straight, flat, or other shape wall as long as the wall fits into the opening 221. Each of the walls 215a, 215b, 215c, 215d form a portion of the edge 216.

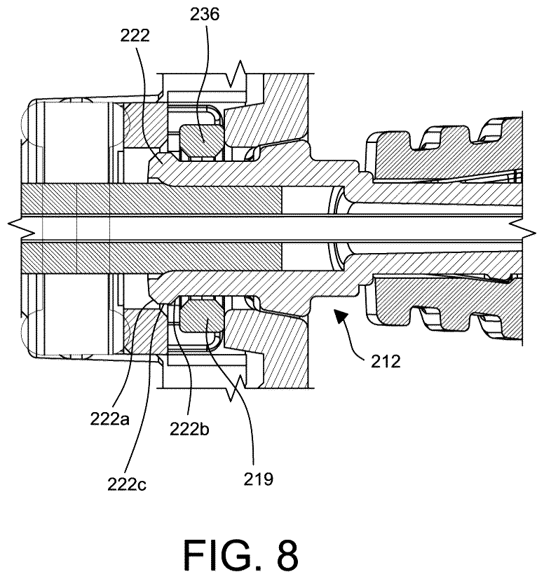

The dilator hub 212 includes a cylindrical portion 217 located between the flange 214 and an annular protrusion 222. In some embodiments, the cylindrical portion 217 is configured as a cylinder having a constant diameter. In other embodiments, the cylindrical portion 217 does not have a constant diameter or is configured as having a shape other than a cylinder, such as a partial cylinder, extruded ellipse, or rectangular prism. In some embodiments, the annular protrusion 222 surrounds the entirety of the cylindrical portion 217. In some embodiments, the annular protrusion 222 surrounds only a portion of the cylindrical portion 217. The annular protrusion 222 is located at a distal end of the cylindrical portion 217 of the dilator hub 212. As shown in FIG. 8, the annular protrusion 222 includes a distal angled surface 222a, a proximal angled surface 222b, and an outer flat surface 222c located between the distal angled surface 222a and the proximal angled surface 222b. The annular protrusion 222 forms a portion of an axial lock feature to restrict axial movement of the dilator 204 relative to the sheath 202, discussed below.

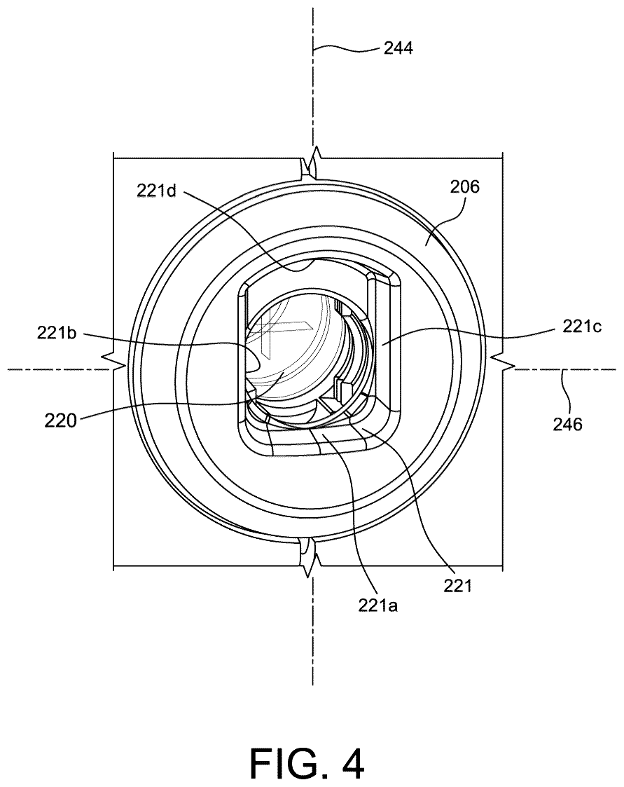

FIG. 4 is a perspective view of the sheath hub 206 of FIG. 2, according to embodiments of the invention. The sheath hub 206 includes an opening 221 having a symmetric shape along a vertical axis 244 and an asymmetric shape along a horizontal axis 246. The vertical axis 244 and the horizontal axis 246 pass through a longitudinal axis of the sheath 202. The opening 221 is configured for coupling to the flange 214 of the dilator 204 to inhibit relative rotation between the sheath 202 and the dilator 204. The opening 221 includes a horizontal bottom wall 221a, a left wall 221b substantially orthogonal to the horizontal bottom wall 221a, a right wall 221c substantially orthogonal to the horizontal bottom wall, and a curved or domed upper wall 221d. In other words, the opening 221 includes a first wall 221a perpendicular to a pair of spaced apart second walls 221b, 221c, and a third convex wall 221d opposite the first wall 221a. The opening 221 surrounds a passageway 220 that is configured to receive the annular protrusion 222 and the cylindrical portion 217 of the dilator hub 212.

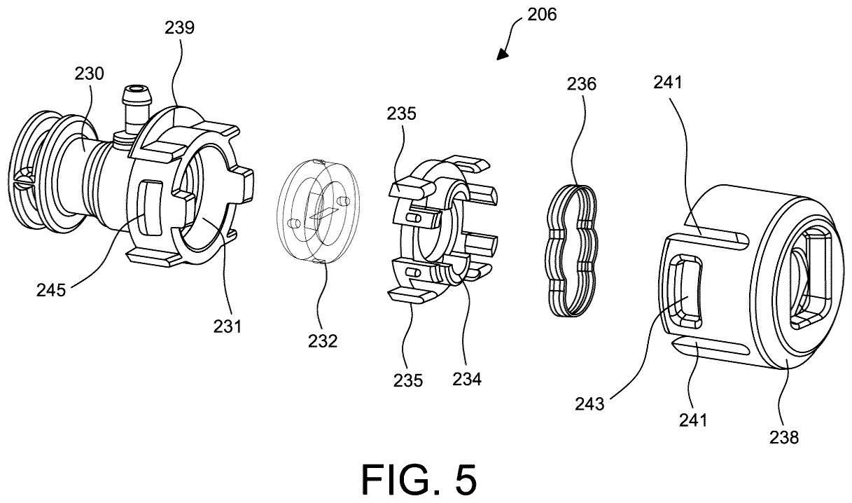

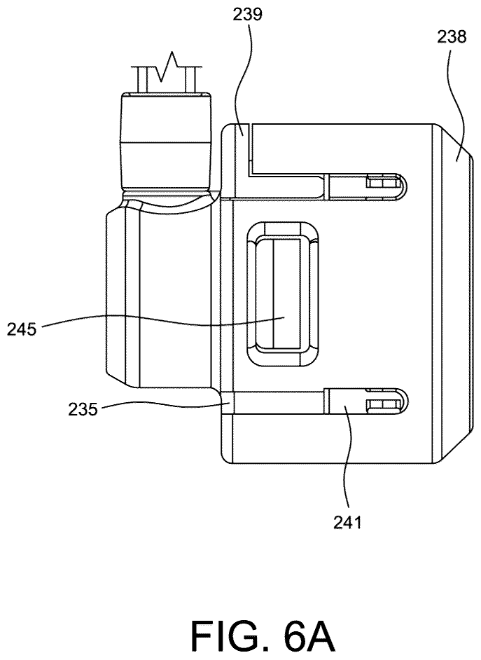

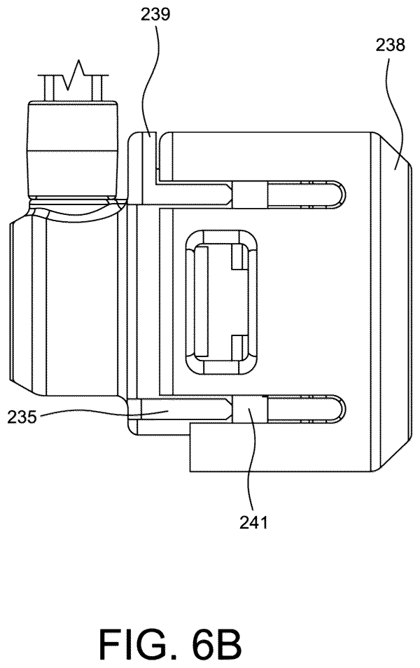

FIG. 5 is an exploded view of the sheath hub 206 of FIG. 2, according to embodiments of the invention. The sheath hub 206 includes a body 230, valve 232, valve plate 234, snaps racetrack 236, a hub cap 238 and a protrusion 239. The body 230 includes a recess 231 which receives the valve 232. The valve 232 is configured to allow for the introduction of a medical device into the sheath 202 while maintaining a seal. The valve plate 234 covers the valve 232 to secure the valve 232 in the recess 231. The valve plate 234 is configured to receive and guide the snaps racetrack 236. The valve plate 234 includes a plurality of rails 235 to guide the hub cap 238. The protrusion 239 prevents incorrect orientation of the hub cap 238 during assembly as shown in FIG. 6B.

FIG. 6A is a side view of the sheath hub 206 of FIG. 2 illustrating a hub cap 238 in a correct orientation, and FIG. 6B is a side view illustrating the hub cap 238 in an incorrect orientation, according to embodiments of the invention. The hub cap 238 includes a plurality of slots 241 configured to receive the plurality of rails 235. During assembly, the valve 232 is located in the recess 231, the valve plate 234 is positioned over the valve 232, the snaps racetrack 236 is positioned within the valve plate 234, and the hub cap 238 is secured to the body 230 to hold the components together. As shown in FIG. 6A, if the hub cap 238 is properly oriented, the plurality of slots 241 slide on the plurality of rails 235 until an opening 243 of the hub cap 238 receives a connector 245 located on the body 230. The opening 243 and the connector 245 can be configured as a snap-fit connection in some embodiments. As illustrated in FIG. 6B, if the hub cap 238 is improperly oriented, the protrusion 239 prevents further movement of the hub cap 238 such that the connector 245 is prevented from being received in the opening 243. This prevents the hub cap 238 from being fastened to the body 230.

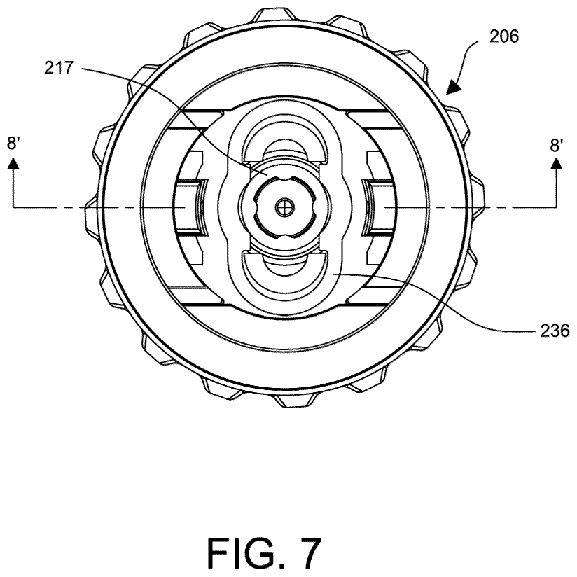

FIG. 7 is a cross-sectional view of the sheath hub 206 mated with the dilator hub 212, according to embodiments of the invention. As illustrated in FIG. 7, the cylindrical portion 217 of the dilator hub 212 is inserted into the passageway 220 of the sheath hub 206. The snaps racetrack 236 is configured to expand as the annular protrusion 222 is moved through the snaps racetrack 236, then contract on the cylindrical portion 217 to form an axial lock feature.

FIG. 8 is a cross-sectional view along line 8′-8′ of the sheath hub 206 mated with the dilator hub 212, according to embodiments of the invention. The annular protrusion 222 is adapted to engage with a corresponding shoulder 219 within the sheath hub 206. The interaction between the protrusion 222 and the shoulder 219 creates a resistance to an axial disengagement force, such that the dilator hub 212 is secured axially within the sheath hub 206. The shoulder 219 is formed by the snaps racetrack 236. Upon insertion of the dilator hub 212 into the sheath hub 206, the snaps racetrack 236 deforms and the annular protrusion 222 enters into a locking chamber 237 as illustrated in FIG. 8. The annular protrusion 222 remains within the locking chamber 237, unable to move proximally past the shoulder 219, in order to axial lock the dilator hub 212 to the sheath hub 206. In one aspect, the locking chamber 237 is configured to allow slight axial movement of the annular protrusion 222 by providing space between the shoulder 219 and the annular protrusion 222. In another aspect, the locking chamber 237 is configured to securely hold the annular protrusion 222, wherein the annular protrusion 222 is in contract with the shoulder 219. The dilator hub 212 and the sheath hub 206 are configured such that when the annular protrusion 222 is positioned within the locking chamber 237, the flange 214 is located within the opening 221 so as to resist or prevent relative rotation as discussed above.

In order to remove the dilator hub 212 from the sheath hub 206, a disengagement force can be created by interaction of the angled surface of the edge 216 with surfaces defining the opening 221. The angled surface of the edge 216 and surfaces defining the opening 221 may have slightly different angles or the same angle (but not oriented horizontally), such that upon application of a torque to the dilator hub 212, an axial disengagement force is generated. The disengagement force (having an axial force component) causes an axial motion of the dilator hub 212, which disengages the dilator hub 212 from the sheath hub 206. When the disengagement force becomes sufficient to overcome an axial lock force formed by an interaction with the annular protrusion 222 and the shoulder 219, relative motion in both axial and rotational directions occurs, allowing removal of the dilator hub 212 from the sheath hub 206. By changing the angles of these surfaces, the amount of torque and the degree of rotation required for disengagement may be adjusted.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

I claim:

1. A dilator for facilitating access to a patient's heart and for coupling with a sheath including a sheath hub, the dilator comprising:
a dilator shaft defining a lumen adapted to receive and support a puncturing device, the dilator shaft includes a proximal portion for manipulation by a user and a tapered distal portion for placement in or near the heart; and
a dilator hub coupled to the proximal portion of the dilator shaft, the dilator hub including a flange having a symmetric shape along a vertical axis and an asymmetric shape along a horizontal axis, the flange configured for coupling to the sheath hub to inhibit relative rotation between the dilator and the sheath.

2. The dilator of claim 1, further comprising an axial lock feature configured to create a resistance to an axial disengagement force, such that the dilator hub is secured axially within the sheath hub.

3. The dilator of claim 2, wherein the axial lock feature includes an annular protrusion adapted to mate with a shoulder.

4. The dilator of claim 3, wherein the protrusion includes a distal angled surface, a proximal angled surface, and an outer flat surface located between the distal angled surface and the proximal angled surface.

5. The dilator of claim 1, wherein the hub includes a cylindrical portion, the axial lock feature being located at a distal end of the cylindrical portion.

6. The dilator of claim 1, wherein the flange includes a thickness that is angled towards the distal portion.

7. The dilator of claim 1, wherein the flange includes a surface that includes a plurality of supports.

8. The dilator of claim 1, wherein the flange includes a horizontal bottom wall, a left wall substantially orthogonal to the horizontal bottom wall, a right wall substantially orthogonal to the horizontal bottom wall, and a curved upper wall.

9. The dilator of claim 1, wherein the flange includes a first wall perpendicular to a pair of spaced apart second walls, and a third convex wall opposite the first wall.

10. A sheath for facilitating access to a patient's heart and for coupling with a dilator including a dilator hub, the sheath comprising:
a sheath shaft defining a lumen adapted to receive and support a dilator, the sheath shaft includes a proximal portion for manipulation by a user and a distal opening through with the dilator can extend; and
a sheath hub coupled to the proximal portion of the sheath shaft, the sheath hub including an opening having a symmetric shape along a vertical axis and an asymmetric shape along a horizontal axis, the opening configured for coupling to a flange of the dilator to inhibit relative rotation between the sheath and the dilator.

11. The sheath of claim 10, wherein the opening includes a horizontal bottom wall, a left wall substantially orthogonal to the horizontal bottom wall, a right wall substantially orthogonal to the horizontal bottom wall, and a curved upper wall.

12. The sheath of claim 10, wherein the opening includes a first wall perpendicular to a pair of spaced apart second walls, and a third convex wall opposite the first wall.

13. The sheath of claim 10, wherein the sheath hub includes a passageway to receive at least a portion of a dilator hub.

14. The sheath of claim 10, wherein the sheath hub includes a recess to receive a valve.

15. The sheath of claim 14, wherein the sheath hub includes at least one connector, and a valve plate configured to receive a snaps racetrack, wherein the valve plate includes a plurality of rails to guide a hub cap and a protrusion.

16. The sheath of claim 15, wherein the protrusion prevents incorrect orientation of the hub cap.

17. The sheath of claim 16, wherein the hub cap includes a plurality of slots to receive the plurality of rails, and at least one opening to receive the connector.

18. A system for facilitating access to a patient's heart, the system comprising:
a sheath having a sheath body defining a lumen adapted to receive a dilator, the sheath body including a proximal portion and a distal portion;
a sheath hub coupled to the proximal portion of the sheath, the sheath hub including an opening;
a dilator having a dilator shaft defining a lumen adapted to receive and support a puncturing device, the dilator shaft including a proximal portion for manipulation by a user and a tapered distal portion for placement in or near the heart; and
a dilator hub coupled to the proximal portion of the dilator shaft, the dilator hub including a flange configured to be received in the opening,
wherein the opening and the flange include a symmetric shape along a vertical axis and an asymmetric shape along a horizontal axis.

19. The system of claim 18, wherein the opening and the flange include a horizontal bottom wall, a left wall substantially orthogonal to the horizontal bottom wall, a right wall substantially orthogonal to the horizontal bottom wall, and a curved upper wall.

20. The system of claim 18, wherein the opening and the flange include a first wall perpendicular to a pair of spaced apart second walls, and a third convex wall opposite the first wall.
